(19) United States
(12) Patent Application Publication
(10) Pub. No.: US 2008/0193994 A1
(43) Pub. Date: Aug. 14, 2008

(54) Systems and methods for the co-treatment of solid organic waste and sewage

(76) Inventors: Chris E. Choate, Discovery Bay, CA (US); James H. Lord, Los Gatos, CA (US)

Correspondence Address: CARR & FERRELL LLP, 2200 GENG ROAD, PALO ALTO, CA, 94303, US

(21) Appl. No.: 11/998,178

(22) Filed: Nov. 27, 2007

Related U.S. Application Data
(60) Provisional application No. 60/861,559, filed on Nov. 27, 2006.

(51) Int. Cl. C12P 5/02; C12M 1/00; C12M 1/02

(52) U.S. Cl. 435/167; 435/290.1; 435/290.2

(57) ABSTRACT

Solid organic waste is processed into a suitable feedstock that can be mixed with sewage for anaerobic digestion. The processing of the solid organic waste initially transforms the solid organic waste into a uniform biomass. The uniform biomass is then subjected to hydrolysis and volatile acid fermentation to dissolve the soluble compounds leaving a small residual solid component. The liquid in which the soluble compounds are dissolved is then mixed with sewage in an anaerobic digester to produce biogas including methane. The residual solid component can be composted.

### CROSS-REFERENCE TO RELATED APPLICATIONS

This application claims the benefit of U.S. Provisional Application No. 60/861,559 filed on Nov. 27, 2006 and entitled “Methods for Adding MSW Biomass into Sewage Treatment Digesters and Anaerobic Digestion Facilities for Performing the Same” which is incorporated herein by reference. This application is also related to U.S. Pat. No. 7,015,028 issued on Mar. 21, 2006 and entitled “Process for Treatment of Organic Waste Materials,” U.S. application Ser. No. 10/954,550 filed on Sep. 29, 2004 and entitled “Systems and Methods for Treatment of Organic Waste Materials,” U.S. patent application Ser. No. 11/031,218 filed on Jan. 6, 2005 and entitled “Organic Waste Material Treatment System,” U.S. patent application Ser. No. 11/385,098 filed Mar. 20, 2006 and entitled “Systems and Processes for Treatment of Organic Waste Materials,” U.S. patent application Ser. No. 11/492,258 filed on Jul. 24, 2006 and entitled “Systems and Processes for Treatment of Organic Waste Materials with a Biomixer,” U.S. patent application Ser. No. 11/584,680 filed Oct. 19, 2006 and entitled “Biomechanical Device for Producing a Biomass,” U.S. patent application Ser. No. 11/343,515 filed on Jan. 30, 2006 and entitled “Process for Generating Useful Biomass from Organic Waste Streams,” and U.S. patent application Ser. No. 11/821,854 filed on Jun. 25, 2007 and entitled “Systems and Methods for Converting Organic Waste Materials into Useful Products,” each of which is incorporated herein by reference.

### BACKGROUND OF THE INVENTION

1. Field of the Invention

The present invention relates generally to disposal of waste materials, and more particularly to the combined anaerobic digestion of organic waste coming from both sewage and municipal solid waste.

2. Description of the Prior Art

Anaerobic digesters are commonly used at municipal sewage treatment facilities to convert organic solids found in sewage into high-energy content gases such as methane. In addition to the value of the methane for producing electricity, anaerobic digestion serves to reduce the amount of residual solids that ultimately have to be disposed of, thereby reducing disposal costs. The residual solids from sewage are known as biosolids or sewage sludge and have to be handled and disposed of according to particular regulations since the residual solids are derived, at least in part, from human and animal wastes.

Anaerobic digesters are designed to handle a maximum expected throughput, but many such digesters used in sewage treatment often operate well below that threshold and therefore operate with excess capacity. In some instances, the maximum expected throughput well exceeds the typical throughput because excess capacity was intentionally built in to accommodate expected growth. In other instances, excess capacity results from the loss of industrial loading when certain producers go out of business or alter their manufacturing processes.

### SUMMARY

An exemplary system of the invention comprises a biomass production system, a hydrolysis reactor, and an anaerobic digester. The biomass production system is configured to convert solid organic waste into a uniform biomass and can include a biomixer and/or a hydropulper for example. The hydrolysis reactor is configured to convert the biomass into residual solids and a liquid including soluble compounds, and in some embodiments includes several tanks operating in parallel. The anaerobic digester is configured to receive sewage and the liquid including soluble compounds and produce biogas therefrom, and in various embodiments comprises either a one-stage or a two-stage digester. The system of the invention can optionally comprise a composting facility to compost the residual solids from the hydrolysis reactor.

An exemplary method of the invention comprises producing a uniform biomass from solid organic waste, producing a residual solid and a liquid including soluble compounds from the biomass by hydrolysis and volatile acid fermentation, and producing biogas from a mixture of sewage and the liquid including soluble compounds by anaerobic digestion. In some embodiments, producing the uniform biomass can include processing the solid organic waste in a biomixer and/or a hydropulper. Producing the residual solid and the liquid including soluble compounds can also comprise recirculating at least some of the liquid including soluble compounds through a hydrolysis reactor. Producing biogas from the mixture of sewage and the liquid including soluble compounds can comprise, in some instances, hydrolysis and volatile acid fermentation of the sewage in a first stage of a two-stage anaerobic digester, and generation of biogas from the liquid including soluble compounds in a second stage of the two-stage anaerobic digester. In some of these embodiments, liquid from the second stage of the two-stage anaerobic digester can be recirculated to the first stage of the two-stage anaerobic digester.

### BRIEF DESCRIPTION OF DRAWINGS

FIG. 1 is a schematic representation of an exemplary facility, according to an embodiment of the present invention.

FIG. 2 is a schematic representation of an exemplary biomass production system, according to an embodiment of the present invention.

FIG. 3 is a schematic representation of an exemplary facility according to another embodiment of the present invention.

FIG. 4 is a flow-chart representation of a method for producing biogas by anaerobically digesting sewage together with the soluble components derived from solid organic waste, according to an exemplary embodiment of the present invention.

### DETAILED DESCRIPTION OF THE INVENTION

The present invention is directed to utilizing the spare capacity of anaerobic digesters such as those used by municipal sewage treatment facilities. According to the present invention, solid organic material from municipal solid waste (MSW) is processed to substantially dissolve the soluble compounds therefrom. These soluble compounds are then fed into an anaerobic digester used to treat sewage in order to utilize any excess digester capacity to thereby increase biogas production. The residual solids from the processing of the solid organic waste can be made into compost.

The present invention is advantageous for several reasons. First, excess anaerobic digestion capacity already exists in may sewage treatment facilities, so the present invention provides for the beneficial conversion of solid organic waste into biogas without the construction of new anaerobic digesters. Secondly, by keeping the insoluble components of the solid organic waste out of the anaerobic digester, these components do not contribute to the biosolids output from the anaerobic digester, and can instead be processed into valuable compost. Thirdly, the operating efficiency of the anaerobic digester increases when the anaerobic digester is operated at or near full capacity. Thus, a greater percentage of the incoming material is converted to biogas and less becomes the biosolid output, and the overall throughput of the anaerobic digester is also increased.

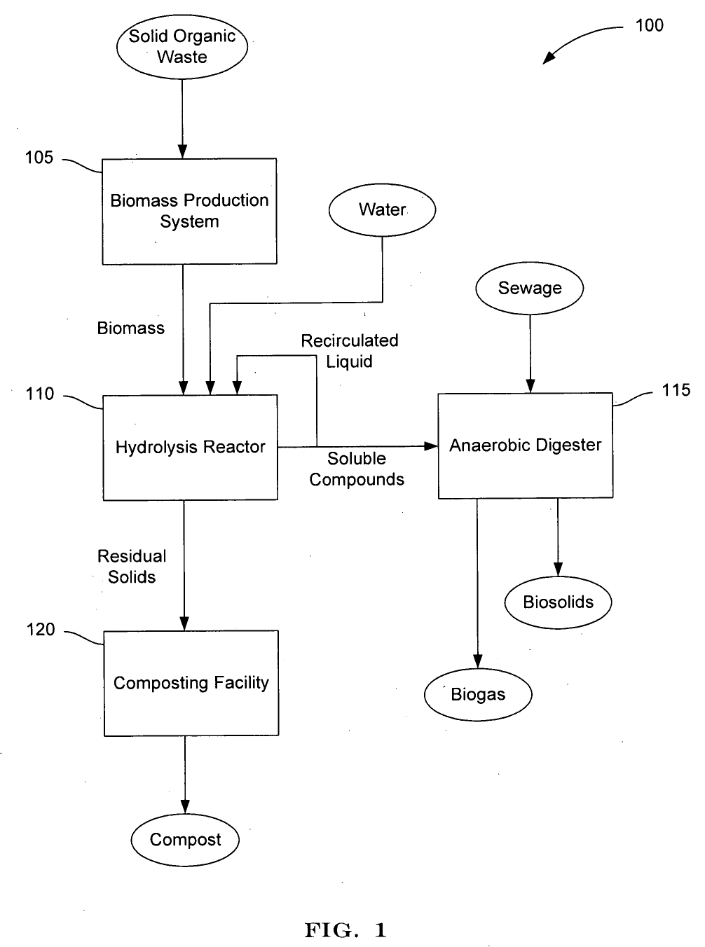

FIG. 1 illustrates a facility 100 for the combined treatment of solid organic waste and sewage. The facility 100 comprises a biomass production system 105, a hydrolysis reactor 110, and a single stage anaerobic digester 115. The biomass production system 105 converts solid organic waste into a uniform biomass that is more suitable for hydrolysis. The hydrolysis reactor 110 converts the biomass into residual solids and a liquid component including soluble compounds. The anaerobic digester 115 receives both the soluble compounds from the hydrolysis reactor 110 and sewage and produces biogas and biosolids. The biogas can include methane which can be converted to electricity to run the facility 100, for example. The biosolids are disposed of by conventional methods such as by landfilling.

The biomass production system 105 can receive the solid organic waste in many forms, from the organic materials within mixed municipal solid waste to source separated organic materials. Specific examples of source separated organic materials include agricultural waste such as corn stover and rice stalks, food waste, yard waste, and paper. The solids content typically ranges from about 30% to about 50% but can be wetter or drier depending on material type and source. Food waste, for example, typically has a low solids content of around 30%, while material high in cellulose such as paper and yard waste tends to have a solids content closer to 50%. It will be understood that as used herein, the term “solid organic waste” can include organic waste having as little as about 30% solids content by weight.

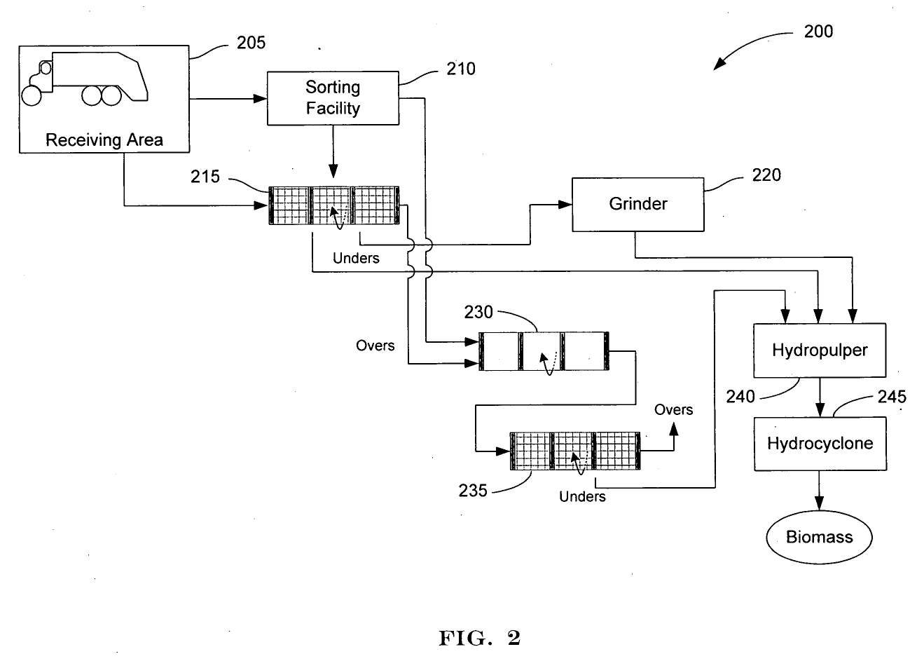

The biomass produced by the biomass production system 105 should be fairly uniform in terms of particle size and composition. In some embodiments, a maximum size of the particles is about 4 inches to facilitate breakdown in the hydrolysis reactor 110. An exemplary biomass production system 105 is described in U.S. patent application Ser. No. 11/821,854 and is shown in FIG. 2. Other suitable biomass production system systems 100 are described in U.S. Pat. No. 7,015,028 and U.S. application Ser. Nos. 10/954,550, 11/031,218, 11/385,098, 11/492,258, 11/584,680, and 11/343,515.

As shown in FIG. 2, a biomass production system 200 comprises a receiving area 205, such as a tipping floor, where the solid organic waste can be delivered to the system 200, for example, by municipal garbage trucks. The system 200 also comprises a sorting facility 210 where various unsuitable materials can be removed from the solid organic waste prior to further processing. The sorting facility 210 can comprise a sorting floor, a sorting line, or both, for example. Depending on the source of the solid organic waste, various degrees of sorting may be employed. A sorting floor is appropriate where little sorting is required, while a sorting line is useful for more significant sorting. For example, MSW is typically directed to the sorting line. On the other hand, some source separated organic waste may require a very limited amount of sorting.

The system 200 further comprises a screening apparatus 215 that can include, for example, a trommel, a screening table, a perforated plate, a disc screen, a finger screen, or a shaker screen. In some instances sorting is unnecessary and the solid organic waste can be moved directly from the receiving area 205 to the screening apparatus 215, bypassing the sorting facility 210. The screening apparatus 215 is configured to screen the solid organic waste into a fraction of the smaller and more desirable “unders” and a residual fraction of “overs.” For some source separated organic waste, such as source separated food waste, the unders from the screening apparatus 215 will include the most organics-rich material, in other words, the material with the highest volatile solids content. The overs, on the other hand, will include more of the less desirable cellulosic material and plastics.

In some instances, the unders from the screening apparatus 215 are directed to a grinder 220 to be ground into a uniform biomass, while the overs can be directed to composting, landfilling, or further processing as described below. An exemplary grinder 220 is a vertical-feed hammer mill. Exemplary final particle size requirements for the uniform biomass produced by the grinder 220 specify a maximum particle size and allow for any size distribution below the maximum, for example, ¾ inch or less, ¼ inch or less, and 1/16 inch or less.

The system 200 also includes a biomixer 230. The biomixer 230 is a biomechanical device described in U.S. patent application Ser. No. 11/584,680. The biomixer 230 employs a combination of mechanical shearing and biological activity in a controlled environment to produce a partially hydrolyzed biomass. In particular, the biomixer 230 includes bacteria capable of facilitating a fermentation process. The bacteria can be any bacteria capable of facilitating a fermentation process, such as aerotolerant anaerobic bacteria. Aerotolerant anaerobic bacteria are specialized anaerobic bacteria characterized by a fermentative-type of metabolism. These bacteria live by fermentation alone, regardless of the presence of oxygen in their environment. Exemplary aerotolerant anaerobic bacteria include species in the genera Desulfomonas, Butyrivibrio, Eubacterium, Lactobacillus, Clostridium, and Ruminococcus.

As shown in FIG. 2, the partially hydrolyzed biomass produced by the biomixer 230 is directed to a screening apparatus 235. The screening apparatus 235 can include a trommel or a screening table, for example. The screening apparatus 235 is configured to screen the partially hydrolyzed biomass into a fraction of unders and a residual fraction of overs. For some source separated organic waste, the unders from the screening apparatus 235 will include the most organics rich material, and the overs will include more of the less desirable cellulosic material and plastics. As with the overs from the screening apparatus 215, the overs produced by the screening apparatus 235 can be directed to composting or a landfill.

The system 200 also comprises a hydropulper 240 including a vessel having an impeller. Exemplary hydropulpers are described in U.S. Pat. Nos. 5,377,917 and 6,379,505 both to Wiljan et al., both incorporated by reference herein. Solid organic waste is mixed with water in the vessel and agitated by the impeller. Through the addition of water, the solids content is reduced in the hydropulper 240 from a typical 25±7% solids content to an 8±2% solids content. Agitation by the impeller creates a slurry and tends to shear paper and plastic materials and otherwise causes a reduction in the particle size of the solids.

Within the hydropulper 240 the heavier materials such a glass, ceramics, stones, and metals tend to sink to the bottom, while lighter materials such as plastics float to the top. The lighter materials can be removed from the hydropulper 240, for example, be skimming the top of the slurry. The heavier materials can be periodically removed from the bottom of the hydropulper 240. The particle size of the solids can be controlled by withdrawing the slurry from a level beneath the level of the lighter fraction and screening the slurry to a typical half inch to one inch size, or less. The larger particles within the slurry that do not pass the screen can be recirculated for additional agitating. The hydropulper 240 produces a slurry with a uniform particle size that is transferred to a hydrocyclone 245. The hydrocyclone 245 is effective to remove grit from the slurry, as also described in U.S. Pat. No. 5,377,917.

Returning again to FIG. 1, the hydrolysis reactor 110 receives the biomass from the biomass production system 105. The biomass within the hydrolysis reactor 110 undergoes hydrolysis and volatile acid fermentation. The hydrolysis breaks down complex organics within the biomass such as cellulous and proteins into smaller soluble molecules. Next, volatile acid fermentation converts the hydrolysis products into organic acids. These processes result in a desirable liquid component that includes soluble compounds such as the organic acids.

In some embodiments, about 80% of the biomass entering the hydrolysis reactor 110 is converted into soluble compounds over a span of about 3 to 5 days. The remaining insoluble materials can be periodically removed from the hydrolysis reactor 110 as residual solids. In some instances it is also possible to extract hydrogen gas from the hydrolysis reactor 110 resulting from the volatile acid fermentation.

Optimal processing within the hydrolysis reactor 110 depends on the specific characteristics of the biomass material, such as composition and particle size. For the benefit of the fast-growing bacteria used within the hydrolysis reactor 110, elevated temperatures and slightly acid conditions are preferred. The hydrolysis reactor 110 can be operated as a batch flow system, a plug flow system, or a complete mix system, for example.

In any of these systems, liquid is flushed through the biomass within the hydrolysis reactor 110 to continuously remove the soluble compounds. Part of the liquid is directed to the anaerobic digester 115 while a recirculating system recirculates the remainder back into the hydrolysis reactor 110. Additional water can be added to the hydrolysis reactor 110 to compensate for the loss of water going to the anaerobic digester 115. In some batch flow embodiments, the hydrolysis reactor 110 comprises several tanks operating in parallel, but staggered in processing time so that each tank discharges residual solids at different times. In these embodiments, the liquid withdrawn from one tank can be introduced into the next so that the liquid circulates through all of the tanks.

The residual solids from the hydrolysis reactor 110 can then be further processed, in some embodiments, and then sent to a composting facility 120 to be converted to a high quality compost. The additional processing can include dewatering and grit removal. While the composting facility 120 can be part of the facility 100, in some embodiments the composting facility 120 represents a separate facility and the residuals solids are hauled from the facility 100 to the composting facility 120.

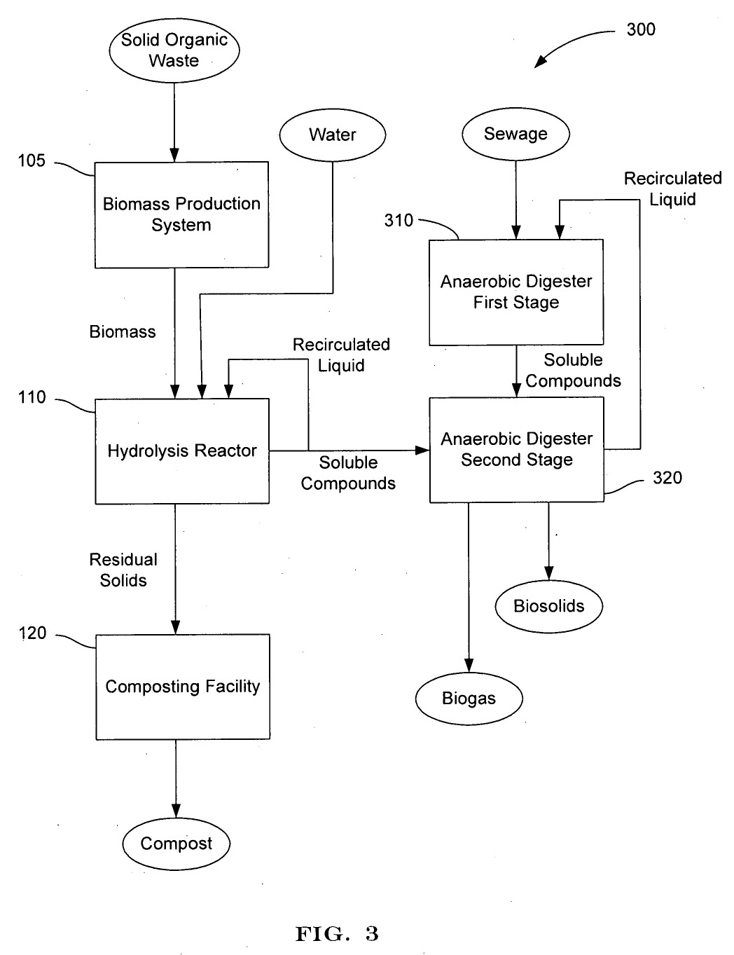

FIG. 3 illustrates another facility 300 for the combined treatment of solid organic waste and sewage. The facility 300 comprises a biomass production system 105, a hydrolysis reactor 110, a two stage anaerobic digester comprising a first stage 310 and a second stage 320, and optionally a composting facility 120. The biomass production system 105, hydrolysis reactor 110, and composting facility 120 operate as described above with respect to FIG. 1. The two-stage anaerobic digester receives sewage into the first stage 310. The first stage 310 operates similarly to the hydrolysis reactor 110 to produce soluble compounds and residual biosolids from the sewage. These soluble compounds, and the soluble compounds from the hydrolysis reactor 110 are directed into the second stage 320 to produce biogas. The biogas can include methane which can be converted to electricity to run the facility 300, for example. Liquid from the second stage 320 can be recirculated back into the first stage 310, in some embodiments. The biosolids from the first stage 310, and any biosolids resulting from the second stage 320, are disposed of by conventional methods.

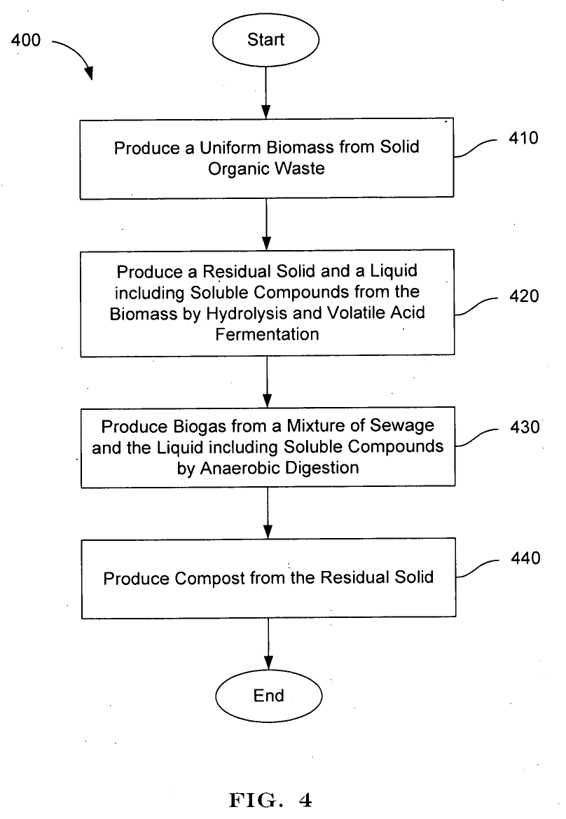

FIG. 4 is a flow-chart representation of an exemplary method 400 of the present invention. The method 400 comprises producing 410 a uniform biomass from solid organic waste, producing 420 a residual solid and a liquid including soluble compounds from the biomass by hydrolysis and volatile acid fermentation, and producing 430 biogas from a mixture of sewage and the liquid including soluble compounds by anaerobic digestion. The method 400 can optionally include producing 440 compost from the residual solid.

In some embodiments, producing 410 the uniform biomass includes processing the solid organic waste in a biomixer and/or a hydropulper. Producing 420 the residual solid and the liquid including soluble compounds can comprise, in some embodiments, recirculating at least some of the liquid including soluble compounds. Where the residual solid and the liquid including the soluble compounds are produced in a hydrolysis reactor, the liquid is recirculated through the hydrolysis reactor.

Producing 430 biogas from the mixture of sewage and the liquid including soluble compounds comprises, in some instances, hydrolysis and volatile acid fermentation of the sewage in a first stage of a two-stage anaerobic digester, and generation of biogas from the liquid including soluble compounds in a second stage of the two-stage anaerobic digester.

In the foregoing specification, the invention is described with reference to specific embodiments thereof, but those skilled in the art will recognize that the invention is not limited thereto. Various features and aspects of the above-described invention may be used individually or jointly. Further, the invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

What is claimed is:

1. A system comprising:
a biomass production system configured to convert solid organic waste into a uniform biomass;
a hydrolysis reactor configured to convert the biomass into residual solids and a liquid including soluble compounds; and
an anaerobic digester configured to receive sewage and the liquid including soluble compounds and produce biogas therefrom.

2. The system of claim 1 wherein the biomass production system includes a biomixer.

3. The system of claim 1 wherein the biomass production system includes a hydropulper.

4. The system of claim 1 further including a recirculation system configured to recirculate at least some of the liquid including soluble compounds back into the hydrolysis reactor.

5. The system of claim 1 wherein the hydrolysis reactor comprises several tanks operating in parallel.

6. The system of claim 1 wherein the anaerobic digester comprises a one-stage digester.

7. The system of claim 1 wherein the anaerobic digester comprises a two-stage digester and a second stage thereof receives the liquid including soluble compounds from the hydrolysis reactor.

8. The system of claim 1 further comprising a composting facility.

9. A method comprising:
producing a uniform biomass from solid organic waste;
producing a residual solid and a liquid including soluble compounds from the biomass by hydrolysis and volatile acid fermentation; and
producing biogas from a mixture of sewage and the liquid including soluble compounds by anaerobic digestion.

10. The method of claim 9 wherein producing the uniform biomass includes processing the solid organic waste in a biomixer.

11. The method of claim 9 wherein producing the uniform biomass includes processing the solid organic waste in a hydropulper.

12. The method of claim 9 wherein producing the residual solid and the liquid including soluble compounds comprises recirculating at least some of the liquid including soluble compounds through a hydrolysis reactor.

13. The method of claim 9 wherein producing biogas from the mixture of sewage and the liquid including soluble compounds comprises hydrolysis and volatile acid fermentation of the sewage in a first stage of a two-stage anaerobic digester, and generation of biogas from the liquid including soluble compounds in a second stage of the two-stage anaerobic digester.

14. The method of claim 13 further comprising recirculating liquid from the second stage of the two-stage anaerobic digester to the first stage of the two-stage anaerobic digester.

15. The method of claim 9 further comprising producing compost from the residual solid.
